(19) United States
(12) Patent Application Publication
(10) Pub. No.: US 2008/0098491 A1
(43) Pub. Date: Apr. 24, 2008

(54) MOUSE MODELS TO STUDY CACHEXIA

(75) Inventor: Zoltan Kiss, Austin, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: ZOLTAN LABORATORIES LLC, Austin, MN (US)

(21) Appl. No.: 11/550,896

(22) Filed: Oct. 19, 2006

(51) Int. Cl. A01K 67/027

(52) U.S. Cl. 800/18

(57) ABSTRACT

Embodiments of the invention provide mouse tumor models involving either the B16 mouse melanoma or MXT mouse mammary tumor, untreated or treated with chemotherapy and/or anti-cachectic agents, for the study of tumor-generated or cancer therapy-generated cachexia-inducing signals and mechanisms. Other embodiments of the invention also provide additional models, including pre-treatment of mouse skin with anti-cachectic proteins prior to tumor implantation, for the study of anti-cachectic signals and mechanisms generated by the skin. To reduce or reverse tumor- and chemotherapy-induced cachexia, embodiments of the invention use the human proteins placental alkaline phosphatase, transferrin, α₁-antitrypsin preparations or combinations thereof as well as chemically synthesized CCDTHT or N,N-diethyl-N-methyl-2-[(9-oxo-9H-thioxanthen-2-yl)methoxy]-ethanaminium iodide or CCDTHT-like compounds.

### FIELD OF THE INVENTION

Embodiments of this invention relate to mouse tumor models treated with commercial or highly purified placental alkaline phosphatase and/or transferrin, and/or α₁-antitrypsin to reduce or reverse tumor- or chemotherapy-induced body weight loss i.e. cachexia. Other embodiments of the invention include a method of studying cachexia using mouse tumor models in which the tumor tissue and chemotherapy can activate catabolic metabolic events in the muscle and adipose tissues as well as mechanisms produced by normal tissues to counteract the cachexia-inducing signals.

### BACKGROUND

Cachexia is a complex metabolic disorder resulting in progressive loss of body weight. Body weight is defined as total weight minus tumor weight. This disorder may result from cancer. Cachexia is responsible for the death of about 20% of cancer patients. Body weight loss also decreases the chance to respond to chemotherapy and renders cachectic patients more prone to toxic side effects. Cachexia affects the metabolism in both skeletal tissue and adipose tissue. Loss of body weight is mainly due to increased lipolysis and decreased lipogenesis in the adipose tissue as well as reduced protein synthesis and increased protein degradation in the skeletal muscle [Gordon, J. N., Green, S. R. and Goggin, P. M. (2005), “Cancer cachexia,” Q. J. Med., 98, 779-788].

The etiology of cachexia is multifactorial; however, there is no consensus on the nature and relative importance of cachexia-inducing factors. In addition, there are yet to be discovered additional factors that contribute to this phenomenon. Finally, it is also not clear what kind of mechanisms exist in the healthy tissues to counter the tumor-derived cachexia-inducing signals.

Cachexia can also be induced by certain therapies such as chemotherapy, radiation therapy and immunotherapy administered for cancer patients. One such chemotherapeutic agent is cisplatin, a widely used platinum-based anti-tumor drug. Chemotherapy can potentially further deteriorate the tumor-induced cachectic state. Like tumor-induced cachexia, the etiology of chemotherapy-induced cachexia also seems to be multifactorial possibly with several participating mechanisms waiting to be identified. Overlapping mechanisms may be involved in both tumor-induced and therapy-induced cachexia. Healthy tissues are likely to produce their own set of signals to counter chemotherapy-induced body weight loss.

Thus, further studies are clearly required to understand the precise mechanisms of cancer cachexia induced by the tumor itself and/or chemotherapy as well as the nature of protective signals produced by the healthy tissue in the vicinity of tumor or elsewhere in the organism. However, such studies are hampered by a sufficient number of suitable experimental models. Good models include a rapidly growing experimental tumor developed in an animal that reduces the observation period. Further, an appropriate model for the study of cachectic signals preferably includes two very different agents that significantly counteract the cachectic effects induced by either the tumor, or chemotherapy, or both. Finally, there is a need for a model in which it is possible to study the anti-cachectic signal(s) generated by the healthy tissue without the influence of tumor tissue.

### SUMMARY OF THE INVENTION

The embodiments of this invention provide various mouse tumor models to study cachexia-inducing signals generated by the tumor and the anti-cachectic signals generated by the healthy tissues.

The animal preferably has an intact immune system, since communication between the tumor and the healthy tissues is likely to involve the immune system. In a suitable model, the growth of tumor itself and/or chemotherapy is associated with cachexia i.e. with loss of body weight defined here as total weight minus tumor weight.

One embodiment of the invention provides a mouse model to study cachexia comprising providing a first mouse and a second mouse having a cachexia-inducing tumor; treating the second mouse with one or more anti-cachectic agents to substantially reduce the cachexia symptoms; and comparing biochemical or cellular changes between the first and second mouse.

In another embodiment, the cachexia-inducing tumor is a B16 mouse melanoma. In another embodiment, the cachexia-inducing tumor is a MXT mouse mammary tumor.

In yet another embodiment of the invention includes placental alkaline phosphatase as the anti-cachectic agent. Other human proteins used as anti-cachectic agents include transferrin and α₁-antitrypsin.

Still other embodiments include a chemically synthesized compound, CCDTHT, or N,N-diethyl-N-methyl-2-[(9-oxo-9H-thioxanthen-2-yl)methoxy]-ethanaminium iodide and CCDTHT-like compounds.

Another embodiment of the invention provides a method for studying cachexia that includes implanting a B16 mouse melanoma or an MXT mouse mammary tumor into a first and second mouse;

administering an anti-cachectic agent to the second mouse;

identifying biochemical or cellular changes induced by the tumor in the first mouse identifying the biochemical or cellular changes induced by the tumor and modified by the anti-cachectic agent in the second mouse; and

comparing the biochemical or cellular changes between the first and second mouse.

In some embodiments of the invention the method further provides identifying biochemical or cellular changes that include detecting changes in cytokines, regulators of food cycle, growth factors, steroids, lipases, mitochondria uncoupling proteins, proteases, transcription factors, transforming growth factor-β family, glucocorticoids, metabolites and enzymes between a first mouse having the B16 mouse melanoma or a MXT mouse mammary tumor and a second mouse having the B16 mouse melanoma or a MXT mouse mammary tumor and treated with an anti-cachectic agent to substantially reduce the cachexia symptoms.

Some embodiments of the invention include comparing biochemical and cellular changes between a mouse model implanted with a MXT mouse mammary tumor and a mouse model implanted with a B16 mouse melanoma tumor.

Another embodiment of the invention includes comparing biochemical and cellular changes between an untreated mouse model implanted with a B16 mouse melanoma tumor and a mouse model treated topically with a protein composition prior to implantation of the B16 mouse melanoma tumor.

### BRIEF DESCRIPTION OF THE DRAWINGS

FIG. 1 shows a digital image of a gel separation, demonstrating that the commercial PALP used for the experiments contains three major bands (lane 2), of which PALP can be separated in a highly purified form (lane 5).

FIG. 2 shows a digital image of a gel separation, demonstrating that the commercial PALP used for the experiments contains three major bands (lane 2), of which α₁-antitrypsin (AT) can be separated in a highly purified form (lane 5).

### DETAILED DESCRIPTION OF THE INVENTION

For the study of mechanisms involved in tumor- and/or cancer therapy-induced body weight loss (cachexia), six suitable in vivo mouse tumor models, each including a specific experimental tumor and a specific anti-cachectic agent are provided by embodiments of the invention. As used herein the term “anti-cachectic proteins or agents” are proteins or chemically synthesized compounds that reduce the loss of body weight. Such proteins include placental alkaline phosphatase (PALP), transferrin (TF) and α₁-antitrypsin (AT), while the chemically synthesized compounds include CCDTHT or N,N-diethyl-N-methyl-2-[(9-oxo-9H-thioxanthen-2-yl)methoxy]-ethanaminium iodide as well as other CCDTHT-like compounds. As used herein the terms “body weight loss” and “cachexia” are used interchangeably, with the “body weight” being defined as total weight minus tumor weight.

Five models use either the B16 mouse melanoma or the MXT mouse mammary tumor, untreated or treated with cisplatin to induce body weight loss, and an anti-cachectic protein or protein composition, or the CCDTHT compound to prevent body weight loss. The sixth model differs from the rest in that the chosen protein or protein composition is topically applied on the skin prior to the implantation of B16 melanoma tumor tissue to study stable anti-cachectic signals generated by the healthy tissue.

In the first model, B16 mouse melanoma tumors are treated with subcutaneously injected commercial PALP, or highly purified PALP, or TF. Commercial PALP preparations contain about 10% PALP, 12% TF, 30% AT, 35% albumin, and degradation products of TF (13%). Both injected commercial PALP (60 mg/kg) and highly purified PALP (14 mg/kg) effectively reverses B16 melanoma tumor-induced body weight loss to comparable extents. On the other hand, injected TF (14 mg/kg) is somewhat less effective. However, depending on the exact goal of the study, all these protein preparations can be used in this model to reduce or decrease tumor-induced body weight loss.

In the second model, B16 mouse melanoma tumor and the surrounding healthy skin is treated topically at regular intervals with a cream containing commercial PALP. Such treatment reverses tumor-induced body weight loss in the absence of significant internal absorption of protein. Thus, this model is especially useful to study biochemical and cellular changes localized to the tumor and surrounding tissues without involving the rest of the body. Comparison of data obtained with this model and the first model is useful to obtain a more complete picture of overall changes in the body induced by the tumor and modified by the protein. Since subcutaneously administered purified PALP and TF also had significant reversal effects on body weight loss in the B16 melanoma model, it is expected that when applied topically singly or in combination they also will exert anti-cachectic effects. Accordingly, in this model, commercial PALP as well as purified PALP, TF and their combination may be used interchangeably to reverse tumor-induced cachexia.

In the third model, B16 mouse melanoma tumors are treated with subcutaneously injected CCDTHT (N,N-diethyl-N-methyl-2-[(9-oxo-9H-thioxanthen-2-yl)methoxy]-ethanaminium iodide), a chemically synthesized agent. Such treatment also results in significant reversal of tumor-induced body weight loss. Data obtained with this model can be compared with data obtained with the first and second models. Such comparison aids to select those biochemical and cellular changes that are common in each model and therefore are the most likely to be involved in the development of cachexia.

In the fourth model, cisplatin-treated MXT mouse mammary tumors are treated with subcutaneously injected purified or commercial PALP or other protein components of the latter such as TF and AT alone or in combination with each other or purified PALP. In this model, cisplatin treatment causes significant reduction in body weight. Treatments of MXT tumor bearing mice with these proteins and particularly with purified PALP results in significant reversal of cisplatin-induced cachexia. This model is suitable to study those biochemical and cellular changes in the tumor and the body's organs that are induced by chemotherapy and that are neutralized by these proteins. Those chemotherapy-induced changes that are prevented by the proteins are the ones that are likely to be responsible for inducing cachexia. Then this set of changes can be compared with those obtained in the previous models to see if tumor-induced and chemotherapy-induced changes involve the same, different, or partially overlapping mechanisms.

In the fifth model, cisplatin-treated MXT mouse mammary tumors are treated with subcutaneously injected CCDTHT compound that causes significant reversal of cisplatin-induced cachexia. This model will also provide a set of biochemical and cellular changes that are induced by cisplatin and prevented or reduced by CCDTHT. Comparison of this set of changes with a similar set of changes obtained with PALP (or TF- or AT)-treated MXT tumor-bearing mice will provide essential help to sort out those common changes that are the most likely to directly relate to cisplatin-induced cachexia.

In the sixth model, the future site of B16 melanoma tumor implantation in the mouse skin is topically pre-treated prior to the tumor implantation with commercial PALP with no additional treatments performed after tumor implantation. When such pre-treatments were performed 6 days and 3 days prior to tumor implantation, tumor-bearing animals gained, instead of lost, body weight. This indicates that commercial PALP, and by implication purified PALP, can generate long-lasting signal(s) in the normal skin that can somehow intercept and neutralize the cachexia-inducing signal(s) generated by the tumor. Using this model for a comparative analysis of untreated and treated skin is expected to facilitate identification of this (or these) anti-cachectic signal(s). Since subcutaneously administered purified PALP and TF also significantly reversed tumor-induced body weight loss in the B16 melanoma model, it is reasonable to assume that when they are used topically, singly or in combination, to pre-treat mouse skin, they also will exert anti-cachectic effects. Accordingly, in this model, commercial PALP as well as purified PALP, TF and their combination may be used interchangeably to reverse tumor-induced cachexia.

### A. Proteins Used for the Prevention of Body Weight Loss.

Embodiments of the invention include using commercial human placental alkaline phosphatase (PALP) suspended in a carrier suitable for topical application on the surface of skin and tumor. In another embodiment the suitable carrier allows subcutaneous injection of commercial PALP around the tumor tissue or directly into the tumor. Commercial PALP used in various embodiments contain about 10% PALP, 12% transferrin (TF), 30% α₁-antitrypsin (AT), 35% albumin, and 13% TF derivatives (proteolytic degradation products of TF) as determined by gel electrophoresis and subsequent sequence analysis. Of these protein, PALP, TF, and AT were found to reduce tumor- or chemotherapy-induced body weight loss. All these proteins, like commercial PALP, are suitable for both topical and injection applications.

Human PALP in solid form is available commercially from, for example, Sigma-Aldrich (St. Louis, Mo.), (Sigma catalog number P3895; CAS Registry Number 9001-78-9). Another commercial source of human PALP is Calbiochem (San Diego, Calif.; catalog number 524604).

The commercial PALP obtained was prepared by the method of Ghosh and Fishman [Ghosh, N. K. and Fishman, W. H. (1968), “Purification and properties of molecular-weight variants of human placental alkaline phosphatase,” Biochem. J., 108, 779-792]. Any PALP preparation that contains well detectable amounts of PALP is suitable for the applications described in embodiments of the invention. PALP preparations that, in addition to PALP, contain TF, AT, albumin and degradation products of these proteins are also suitable. For some models, PALP was highly purified from commercial (Sigma-Aldrich) PALP by a slightly modified method described earlier [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167].

The human alkaline phosphatase family also includes the tissue non-specific (liver/bone/kidney) alkaline phosphatase, the intestinal alkaline phosphatase, and the PALP-like (germ cell) alkaline phosphatase. Substantially purified preparations of intestinal, tissue non-specific, and germ cell alkaline phosphatase enzymes are all available commercially (for example, from Sigma-Aldrich). Appropriate purification methods are known for their isolation from human blood, liver, and other organs. Also, recombinant forms of each of these alkaline phosphatases have already been produced.

To simplify the text, as used herein, the term “PALP” and the phrase “human PALP” are used interchangeably to refer to human alkaline phosphatases including placental alkaline phosphatase. The phrase “active PALP” means the human alkaline phosphatase proteins and their glycosylated and non-glycosylated forms as well as peptides derived from these proteins that, when administered locally or by an injection method, appreciably reduce lean body weight loss induced by a tumor and/or chemotherapy.

PALP is a member of the alkaline phosphatase group of enzymes that hydrolyzes phosphate-containing compounds at alkaline pH. Mature PALP is a dimer of two identical glycosylated subunits. Each subunit has an approximate molecular weight of 66 kDa, as determined by gel electrophoresis.

It was reported earlier that PALP can enhance both the proliferation and survival of mouse embryo fibroblasts as well as fibroblast-like cells derived from the lung of human fetus [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167; Q.-B. She, J. J. Mukherjee, T. Chung, and Z. Kiss (2000), “Placental alkaline phosphatase, insulin, and adenine nucleotides or adenosine synergistically promote long-term survival of serum-starved mouse embryo and human fetus fibroblasts,” Cellular Signalling, 12, 659-665]. As it was reported earlier, (in U.S. patent application Ser. No. 11/463,022, filed on Aug. 8, 2006, entitled “Use of alkaline phosphatase to maintain healthy tissue mass in mammals,” to Zoltan Kiss) PALP exerts inhibitory effects on tumor growth in various tumor models.

In various embodiments of the present invention, commercial or highly purified PALP is used in several tumor model systems to prevent body weight loss induced by either a tumor or chemotherapy. As it was already defined above, “body weight” is obtained by subtracting tumor weight from total weight.

The exact mechanism(s) by which PALP prevents tumor- and chemotherapy-induced cachexia is (are) presently unknown. However, stable anti-cachectic signals are clearly generated in tumor-free skin, because in one model pre-treatment of skin with PALP prior to tumor implantation reversed body weight loss induced later by the tumor. This PALP-treated model is suitable to help investigators to identify the anti-cachectic signals generated by healthy tissues.

Digestion of PALP with the protease bromelain was shown to yield an active derivative [U.S. patent application Ser. No. 10/653,622, filed Sep. 2, 2003 and entitled “Use of Placental Alkaline Phosphatase to Promote Skin Cell Proliferation”; Pub. No. US2005/0048046 A1, Pub. Date, Mar. 3, 2005]. Consequently, one who is skilled in the art may synthesize or develop an active derivative that is a smaller fragment of a PALP amino acid sequence and demonstrates efficient anti-cachectic effects similar to that obtained with native PALP. By way of example, modification of a PALP amino acid sequence or a sequence of smaller PALP peptides by exchanging amino acids at critical sites to yield an active derivative may also result in the maintenance or even improvement of the beneficial effects of PALP. Likewise, chemical or enzymatic changes in the level and position of glycosylation may maintain or enhance the effects of PALP or its derivatives. In embodiments of the invention, modified PALP, smaller PALP-derived peptides, or modified PALP-derived peptides may be similarly effective, and are each considered to be active derivatives. Likewise, PALP isolated from placenta tissue or produced in recombinant form is considered to be similarly effective in preventing tumor- or chemotherapy-induced cachexia.

Human PALP, and particularly a smaller molecular mass active derivative, may also be obtained by chemical synthesis using conventional methods. For example, solid-phase synthesis techniques may be used to obtain PALP or an active derivative.

Recombinant methods to obtain quantities of PALP (and active derivative) are also suitable. Since cDNA of PALP is available, recombinant protein can be produced by one of the many existing conventional methods for recombinant protein expression. PALP has been cloned and overexpressed in a mammalian cell line as described by Kozlenkow et al. [Kozlenkow, A., Manes, T., Hoylaerts, M. F. and Millan, J. L. (2002), “Function assignment to conserved residues in mammalian alkaline phosphatase,” J. Biol. Chem., 277, 22992-22999]. Production of recombinant PALP by bacteria [Beck, R. and Burtscher, H. (1994), “Expression of human placental alkaline phosphatase in Escherichia coli,” Protein Expression and Purification, 5, 192-197] and yeast [Heimo, H., Palmu, K. and Suominen, I. (1998), “Human placenta alkaline phosphatase: Expression in Pichia pastoris, purification and characterization of the enzyme,” Protein Expression and Purification, 12, 85-92] has also been reported.

Bacterial expression yields non-glycosylated PALP. Embodiments of the present invention include using native glycosylated PALP and its active derivatives as well as non-glycosylated PALP and its active derivatives.

As indicated earlier, the commercial PALP preparation contains other proteins that may be removed or may be retained depending on the model used. Commercial PALP preparations can be used as starting material to obtain homogeneous PALP, transferrin (TF) and α₁-antitrypsin (AT) by successive chromatographic steps, as described in detail in Example 1. In certain models TF and AT may also be used to partially inhibit the cachexia-inducing effects of tumor and chemotherapy.

It is conceivable that certain PALP preparations, with or without TF and AT, may also be obtained by extraction from placental tissue followed by different purification steps. Any placental preparation that contains sufficient amount of PALP that exerts a well measurable anti-cachectic effect may be used in various embodiments of the invention so long as the other proteins and compounds present in the preparation do not interfere with the anti-cachectic actions of PALP. In some embodiments, like in the commercial PALP preparation, PALP may not be the dominating component. In other embodiments, highly purified or substantially purified preparations may be used. A highly purified or substantially purified PALP preparation contains less than 5% of other proteins. Substantially purified PALP is obtained from a starting material by one or more purification steps (such as solvent extraction, column separation, chromatographic separation, or techniques known to one skilled in the art) that enrich the concentration of PALP to an extent that PALP is the dominating component. The term “substantially purified” should not be construed to connote absolute purity.

The stimulatory effect of PALP on fibroblast proliferation in vitro is enhanced by pre-heating it at 65-75° C. for 30 minutes [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167]. It is reasonable to expect that pre-heating of PALP at 65-75° C. prior to its use may also enhance its anti-cachectic effect. Thus, a step of heat-activation of both commercial and highly purified PALP preparation may be included prior to their applications.

The stimulatory effect of PALP on fibroblast proliferation in vitro is also enhanced by adding calcium and zinc to the medium [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167]. This may reflect that PALP has binding sites for both zinc and calcium required for its biological activity, and that during protein preparation PALP may partially become metal deficient. Regardless of the mechanism, the final preparations of both commercial and highly purified PALP may include 1-3 mM of a calcium containing compound (for example, calcium chloride) and/or 1-50 μM of a zinc containing compound (for example, zinc chloride or zinc sulfate).

TF, a component of commercial PALP, may also be used alone in the tumor models to partially prevent tumor- or chemotherapy-induced body weight loss.

TF is a glycoprotein with an approximate molecular weight of 80 kDa. Its major function is to carry iron from the sites of intake into the systemic circulation to the cells and tissues. Since iron is essential for cell function, TF serves as a growth factor for many cell types including cancer cells; for this reason, it is a standard component of several growth media used for cell culture. In embodiments of this invention both iron-saturated and iron-free TF are used.

In several patent applications and patents, TF has been listed as a minor component of complex mixtures that may add to the effects of major promoters of skin rejuvenation and repair without either specifying or directly proving such role [U.S. patent application Ser. No. 10/222,949, filed Apr. 10, 2003 and entitled “Composition and Methods for Skin Rejuvenation and Repair”; U.S. Pat. No. 5,461,030, issued Oct. 24, 1995 and titled “Compositions and Methods for Enhancing Wound healing”; U.S. Pat. No. 5,591,709, issued Jan. 7, 1997 and entitled “Compositions and Methods for Treating Wounds”; U.S. Pat. No. 5,556,645, issued Sep. 17, 1996 and entitled “Methods of Enhancing Wound Healing and Tissue Repair”; U.S. Pat. No. 4,347,841, issued Sep. 7, 1982 and titled “Biological Wound Covering and Method for Producing Same”], all incorporated herein by reference. However, TF has never been used as an anti-cachectic agent to aid identification of signaling mechanisms involved in body weight loss. Also, none of the above publications would be expected to lead someone to conclude that TF may reduce tumor- or chemotherapy-induced body weight loss.

As used herein, the term “TF” and the phrase “human TF” are used interchangeably to refer to transferrin. As used herein, “active TF” means the human protein, or closely related mammalian proteins, and its/their glycosylated and non-glycosylated forms as well as peptides derived from these proteins that are effective to reduce body weight loss induced by a tumor or chemotherapy. Examples of suitable preparation of human TF used are from Sigma-Aldrich with the following specifications: (i) catalog number, T 3309; at least 98% pure; 300-600 μg iron per gram protein, (ii) catalog number, T 2036; at least 97% pure; substantially iron-free (apo-TF), and (iii) catalog number, T 0665, at least 97% pure; 1100-1600 μg iron per gram protein (holo-TF). Similarly, any other commercial preparation that contains sufficient amounts of TF to reduce cachexia is suitable for use in embodiments of the invention.

Because TF is a major component of human blood, and placenta always contains significant volume of blood, the placenta tissue is also a potential source for the isolation of this protein. Chromatographic separation methods are available for the purification of TF from raw extracts of blood and/or placenta. For example, it is possible to enrich TF, along with some other glycoproteins such as PALP and AT, using a so-called Concanavalin-A-Sepharose column, which separates glycoproteins based on their ability to interact with lectins such as Concanavalin-A. This step is then may be followed by other column chromatography methods, such as size-exclusion chromatography, to separate glycoproteins from each other. As a result, the purified TF preparation will have a higher concentration of TF than found in a raw tissue or blood extract. The term “purified TF” is used herein to encompass compositions that are obtained from a starting material by one or more purification steps (such as solvent extraction, column separation, chromatographic separation, or techniques known to one skilled in the art) that enrich the concentration of TF, relative to the starting material. The term “highly purified TF” is used herein to encompass a preparation that contains less than 5% of other proteins. The term “highly purified TF” should not be construed to connote absolute purity of the protein.

TF is also present in significant amounts in the commercial PALP preparation. Accordingly, TF can also be isolated in a highly purified state along with PALP and AT using either commercial PALP or placenta as the starting material.

The sequence of human TF is known and the corresponding cDNA is available. This allows expression of original TF or its point and deletion mutants in any cell line of choice, for example in insect cells [Tomiya, N., Howe, D., Aumiller, J. J., Pathak, M., Park, J., Palter, K. B., Jarvis, D. L., Betenbaugh, M. J. and Lee, Y. C. (2003), “Complex—type biantennary N-glycans of recombinant human transferring from Trichoplusia in cells expressing mammalian β-1,4-galactotransferase and β-1,4-N-acetylglucosaminenyltransferase II,” Glycobiology, 13, 23-34]. These and similar techniques may be used to generate, at larger scale, various active recombinant forms of TF and its derivatives.

The stimulatory effects of TF on fibroblast proliferation in vitro are not altered by pre-heating it at up to 75° C. for 30 min. Thus, pre-heating of TF-containing compositions to enhance the effects of other components, such as PALP and AT will unlikely to alter the ability of TF to reduce tumor- and chemotherapy-induced body weight loss.

Both iron-free and iron-containing TF to decrease body weight loss may be used in various embodiments of the invention.

As used herein, the term “AT” and the phrase “human AT” are used interchangeably to refer to α₁-antitrypsin. As used herein, active AT means the various isoforms of the human protein, or closely related mammalian proteins, and its/their glycosylated and non-glycosylated forms as well as peptides derived from these proteins that can reduce or prevent body weight loss induced by a tumor or chemotherapy.

AT (in the literature also often called al-proteinase inhibitor) belongs to the large family of serine protease inhibitors, or serpins, that act as irreversible suicide inhibitors of proteases [Janciauskiene, S. (2001), “Conformational properties of serine proteinase inhibitors (serpins) confer multiple pathophysiological roles,” Biochem. Biophys. Acta, 1535, 221-235]. While AT is a particularly effective inhibitor of elastase, it also inhibits other proteases such as trypsin.

AT also exerts effects on cell proliferation that may be independent of its antiprotease actions. In particular, AT was shown to stimulate proliferation of astrocytes and fibroblasts [referenced in Janciauskiene, S. (2001), “Conformational properties of serine proteinase inhibitors (serpins) confer multiple pathophysiological roles,” Biochem. Biophys. Acta, 1535, 221-235].

Relatively pure AT is commercially available (for example, from Sigma-Aldrich; catalog number: A 9024), and it also can be highly purified from commercial PALP preparation which contains it as a significant “contaminant”. Purified AT, purified from commercial PALP preparation (Sigma-Aldrich) by a previously described method [She, Q.-B., Mukherjee, J. J., Crilly, K. S. and Kiss, Z. (2000), “α₁-Antitrypsin can increase insulin-induced mitogenesis in various fibroblast and epithelial cell lines,” FEBS Lett., 473, 33-36] was used. Purification of AT is also described under “Examples”. By implication, AT can be isolated in essentially pure form from human placenta. Placenta not only produces this protein [Bergman, D., Kadner, S. S., Cruz, M. R., Esterman, A. L., Tahery, M. M., Young, B. K. and Finlay, T. H. (1993), “Synthesis of α₁-antichymotrypsin and α₁-antitrypsin by human trophoblast,” Pediatric Res., 34, 312-317], but placenta-associated blood also is a rich source of AT.

The sequence of AT is known and the corresponding cDNA as well as appropriate molecular biology techniques are available to produce recombinant forms of AT [Leicht, M., Long, G. L., Chandra, T., Kurachi, K., Kid, V. J., Mace, M. Jr., Davie, E. W. and Woo, S. L. C. (1982), “Sequence homology and structural comparison between the chromosomal human α₁-antitrypsin and chicken ovalbumin genes,” Nature, 297, 655-659; Long, G. L., Chandra, T., Woo, S. L. C., Davie, E. W. and Kurachi, K. (1984), “Complete Sequence of the cDNA from human α₁-antitrypsin and the gene for the S variant,” Biochemistry, 23, 4828-4837].

The stimulatory effects of AT on fibroblast proliferation in vitro is enhanced by pre-heating it at 65-75° C. for 30 min or at 41° C. for 21 hours [She, Q.-B., Mukherjee, J. J., Crilly, K. S. and Kiss, Z. (2000), “α₁-Antitrypsin can increase insulin-induced mitogenesis in various fibroblast and epithelial cell lines,” FEBS Lett. 473, 33-36]. Thus, it is reasonable to assume that other biological effects of AT, such as reduction of tumor- or chemotherapy-induced cachexia, are either also enhanced or at least are not altered by heat treatment. Therefore, a step of heat-activation of AT may be included during the preparation of active compositions.

AT preparations that are commercially available contain impurities. Impure commercial AT preparations can be used as starting material to obtain homogeneous AT by successive chromatographic steps, as described in detail in Example 2. Impure AT preparations may also be used in the tumor models so long as the impurities are not toxic and do not interfere with the ant-cachectic effect of AT.

A preparation containing human AT may also be obtained by extraction from placental tissue that synthesizes the protein during pregnancy. By way of example, a preparation may be obtained by butanol extraction of homogenized placenta. Other methods of extraction from placental tissue are also suitable.

If blood- or placenta-derived AT preparation is to be used in the practice of embodiments of the present invention, a raw extract or fraction should be treated to enrich the concentration of AT and obtain a purified preparation. A purified preparation will have a higher concentration of the active component than found in a raw tissue or blood extract. The term “purified” is used herein to encompass compositions that are obtained from a starting material by one or more purification steps such as solvent extraction, column separation, chromatographic separation or techniques known to one skilled in the art that enhance the concentration of AT, relative to the starting material. The term “purified AT” should not be construed to connote absolute purity of the protein.

The B16 Mouse Melanoma Tumor.

The highly metastatic B16 mouse melanoma tumor is a frequently used experimental tumor model [see, for example, Rose, M. L., Madren, J., Bunzendahl, H. and Thurman, R.G. (1999), “Dietary glycine inhibits the growth of B16 melanoma tumors in mice,” Carcinogenesis, 20, 793-798]. It grows rapidly (which decreases the observation period) and also induces loss of body weight. These two latter properties were important for their selection for some of the models used in embodiments of this invention. A further advantage is that this tumor can be developed in immune competent mice, i.e. in mice that have an intact immune system. B16 tumors are used in embodiments of the invention to produce cancer cachexia. Implantation, maintenance, and treatment of B16 tumors are described in detail in “Example 3.” In U.S. application Ser. No. 11/465,876, filed Aug. 21, 2006, entitled “Compositions to Reduce or Prevent Skin Cancer,” incorporated herein by reference, the B16 mouse melanoma model was used to demonstrate that both highly purified placental alkaline phosphatase and commercial placental alkaline phosphatase reduced tumor growth when administered subcutaneously or topically. In the B16 model, placental alkaline phosphatase was also shown to enhance the effect of CCDTHT, a chemotherapeutic agent, on tumor growth.

The Cisplatin-Treated MXT Mouse Mammary Tumor.

The estrogen receptor positive MXT mouse mammary tumor is another tumor model [see, for example, Szepeshazi, K., Schally, A. V., Halmos, G., Lamharzi, N., Groot, K. and Horvath, J. E. (1997), “A single in vivo administration of bombesin antagonist RC-3095 reduces the levels and mRNA expression of epidermal growth factor receptors in MXT mouse mammary cancers,” Proc. Natl. Acad. Sci. USA, 94, 10913-10918]. It grows rapidly with a parallel decrease in body weight of the host. This tumor can also be developed in immune competent mice. Implantation, maintenance, and treatment of MXT tumors are described in detail in “Example 3.” In U.S. application Ser. No. 11/463,022, filed Aug. 8, 2006, entitled “Use of Alkaline Phosphatase to Maintain Healthy Tissue Mass in Mammals,” incorporated herein by reference the human HT 168 melanoma model was used to demonstrate that placental alkaline phosphatase reduces tumor induced loss of total weight (which includes tumor weight). Purified placental alkaline phosphatase was also shown to reduce total weight loss in the cisplatin treated mouse MXT tumor model.

Cisplatin is a widely used highly cytotoxic chemotherapeutic agent. In case of breast cancer patients, it is usually used in combination with other anti-cancer agents [Orlando, L., Colleoni, M., Curigliani, G., Nole, F., Ferretti, G., Masci, G., Peruzzotti, G., Minchella, I., Intra, M., Veronesi, P., Viale, G. and Goldhirsch, A. (2001), “Chemotherapy with vinorelbine, cisplatin and continuous infusion of 5′fluoroacil in locally advanced breast cancer: A promising low-toxic regimen,” Anticancer Res., 21, 4135-4140; Kariya, S., Ogawa, Y., Nishioka, A. and Yoshida, S. (2002), “Docetaxel-cisplatin combined chemotherapy in Japanese patients with anthracycline-pretreated advanced breast cancer,” Oncol. Rep., 9, 1345-1349].

Cisplatin very reproducibly induces severe body weight loss in the MXT tumor model. Thus cisplatin-treated MXT tumor combined with the use of anti-cachectic proteins and CCDTHT provides a suitable experimental system to study the mechanisms involved in the development of chemotherapy-induced cachexia.

Mouse Tumor Models to Study the Cachectic Signals Generated by the Tumor and/or Chemotherapy.

Presently there is an uncertainty over what causes cachexia. One theory holds that pathological alterations in the food cycle regulated by nutrients and feeding-stimulatory peptides such as Neuropeptid Y and leptin, is deregulated in the hypothalamic orexigenic network leading to decreased energy intake coupled with high metabolic demand for nutrients. Another theory is that tumor-derived factors initiate and maintain the cachectic syndrome. In line with such possibility, a proteolysis-inducing factor (PIF) was isolated from cachectic cancer patients which upregulates the ubiquitin-proteasome proteolytic pathway and thereby induces protein breakdown in the muscle. Tumors also produce a lipid-mobilizing factor (zinc α2-glycoprotein) which stimulates adipose tissue breakdown. In addition, increased expression of various cytokines (tumor necrosis factor-α, interleukin-6, and interleukin-1β), mitochondria uncoupling proteins, prostaglandin, myostatin and angiotensin II have all been implicated in the maintenance of cachexia [Tisdale, M. J. (2005), “Molecular pathways leading to cancer cachexia,” Physiology, 20, 340-348; Gordon, J. N., Green, S. R. and Goggin, P. M. (2005), “Cancer cachexia,” Q. J. Med., 98, 779-788, and references therein; Sherry, B. A., Gelin, J., Fong, Y., Marano, M., Wei, H., Cerami, A., Lowry, S. F., Lundholm, K. G. and Moldawer, L. L. (1989), “Anticachectin/tumor necrosis factor-α antibodies attenuate development of cachexia in tumor models,” FASEB J., 3, 1956-1962; Tamura, S., Ouchi, K. F., Mori, K., Endo, M., Matsumoto, T., Eda, H., Tanaka, Y., Ishitsuka, H., Tokita, H. and Yamaguchi, K. (1995), “Involvement of human interleukin 6 in experimental cachexia induced by a human uterine cervical carcinoma xenograft,” Clin. Cancer Res., 1, 1353-1358; Lazarus, D. D., Destree, A. T., Mazzola, L. M., McCormack, T. A., Dick, L. R., Xu, B., Huang, J. Q., Pierce, J. W., Read, M. A., Coggins, M. B., Solomon, V., Goldberg, A. L., Brand, S. J. and Elliott, P. J. (1999), “A new model of cancer cachexia: contribution of the ubiquitin-proteasome pathway,” Am. J. Physiol., 277, E332-E341; Davis, T. W., Zweifel, B. S., O'Neal, J. M., Heuvelman, D. M., Abegg, A. L., Hendrich, T. O. and Masferrer, J. L. (2004), “Inhibition of cyclooxygenase-2 by celecoxib reverses tumor-induced wasting,” J. Pharmacol. Exp. Therap., 308, 929-934]. Accordingly, biochemical and cellular changes, induced by the tumor and modified by the anti-cachectic agent, will be identified, such as various cytokines regulators of food cycle, growth factors, steroids, lipases, mitochondria uncoupling proteins, proteases, transcription factors, transforming growth factor-β family, glucocorticoids, metabolites and enzymes.

Model 1. Treatment of B16 Melanoma with Subcutaneously Injected PALP or TF.

In this model, B16 mouse melanoma tumors are developed as described in Example 3, and regularly treated with commercial PALP or highly purified PALP or TF via subcutaneous injection in the vicinity (within 1-cm distance) of the tumor. The proteins are suspended in a suitable carrier, such as physiological saline, prior to injection. However, any other carrier which has no biological effects on its own and which does not interfere with the actions of proteins is suitable. The stock solution contains 10-100 mg protein per ml; the less purified the protein preparation the more of it is needed for the stock solution. In embodiments of the invention, 50 μl of a stock solution of commercial PALP solution containing 30-mg protein per 1-ml physiological saline is injected. In case of purified PALP and TF, the same volume of stock solutions containing 6-8-mg of protein per 1-ml is injected.

The treatments may start anytime between 7-20 days after tumor implantation depending on the research goal. The treatments may be repeated daily or may be administered every second or third day. For maximum effectiveness, it is recommended that treatments be performed at least every second day.

Tissue samples from the tumor, surrounding tissue or any other tissue may be taken for the analysis of biochemical and cellular changes at any time during the treatment and observation period. However, it is recommended that from one animal only one sample be taken. This model provides an opportunity to compare biochemical and cellular changes in the tumor and other tissues in the untreated and protein-treated mice. It is expected that treatment with the proteins will down-regulate the activity and/or expression of specific cell components (probably with signaling function) characteristic of the tumor tissue. This would lead one to consider the role of these components in the induction of cachexia. Then agents and methods could be designed to block the actions of these cachexia-inducing components/signals even more effectively than PALP and TF do. Analysis of tumor- and protein-induced biochemical and cellular changes in the tumor and healthy tissues as permitted by this model is expected to further help understanding the mechanism of cachexia.

Model 2. Topical Treatment of B16 Melanoma with PALP.

In this model, B16 mouse melanoma tumors are developed as described in Example 3, and regularly treated topically with either commercial PALP, or a PALP preparation that also contains TF or TF plus AT, or highly purified PALP. The proteins are dispersed in a suitable carrier for topical application. In embodiments of the invention, Vaselinum cholesteratum was used as the carrier. However, any other carrier which has no biological effects on its own, which does not interfere with the actions of proteins, and which can be applied on the skin in a stable manner is suitable. The concentration of protein in the carrier is 0.25 to 10-mg per 1-g carrier. In some embodiments, 4 mg commercial PALP is dispersed in 1-g Vaselinum cholesteratum. In case of highly purified PALP, 0.2 to 1-mg protein may be dispersed per 1-g of Vaselinum cholesteratum. In other embodiments, 150-200 mg PALP-containing cream is applied on the tumor surface and/or on the surrounding skin's surface depending on the goal of the study. The treatments may start anytime between 5-20 days after tumor transplantation depending on the research goal. The treatments may be repeated daily or may be administered every second or third day. For maximum effectiveness, it is recommended that treatments be performed once every day.

Since subcutaneously administered TF also reduces tumor-induced body weight loss it can be reasonably expected that topically applied TF can elicit similar effects. For topical application, some embodiments include 0.2 to 1-mg purified TF may be dispersed per 1-g of Vaselinum cholesteratum.

Tissue samples from the tumor and/or the surrounding tissue or any other tissue may be taken for the analysis of biochemical and cellular changes at any time during the treatment/observation period. It is recommended that from one animal only one sample be taken.

This model is different from model 1 in that it provides an opportunity to compare biochemical and cellular changes localized to the tumor and the surrounding skin tissue. Because PALP is a large protein, when administered topically its absorption into internal organs is negligible. Therefore, topically applied PALP is not expected to induce any significant biochemical or cellular change in the internal organs. This is a great help to separate strictly tumor-generated cachexia-inducing signal from a much larger number of signals that may be generated by internal organs in response to the tumor and PALP, many of them probably unrelated to the induction of cachexia.

The fact alone that topical application of PALP exhibits anti-cachectic effect is a proof that such application method is effective in producing signals that can counter the tumor-derived cachectic signals. Additional evidence for topically applied PALP being able to induce changes in the tumor is provided in Example 6. In that Example, topically applied PALP is shown to significantly stimulate expression of mRNA for certain cytokines.

Model 3. Treatment of B16 Melanoma with Subcutaneously Injected CCDTHT.

In this model, B16 mouse melanoma tumors are treated with subcutaneously injected CCDTHT, a chemically synthesized agent, or a similar compound. In U.S. patent application Ser. No. 11/458,502, filed Jul. 19, 2006, entitled “Compounds and compositions to control abnormal cell growth” to Zoltan Kiss, incorporated herein by reference, the structures and methods of chemical synthesis of CCDTHT and CCDTHT-like compounds have been described. The primary anti-cachexia compound used in this model is CCDTHT or N,N-diethyl-N-methyl-2-[(9-oxo-9H-thioxanthen-2-yl)methoxy]-ethanaminium iodide. All CCDTHT-like compounds disclosed in the above patent application, including [3-(3,4-Dimethyl-9-oxo-9H-thioxanthen-2-yloxy)-2-hydroxypropyl]trimethyl-ammonium chloride and N,N,-diethyl-N-methyl-3-(2-methyl-9H-thioxanthen-9-ylidene)-propan-1-aminium-iodide, are suitable based on their similar anti-tumor activities.

CCDTHT is dissolved in a suitable carrier, such as physiological saline, prior to injection. However, any other carrier which has no biological effects on its own and which does not interfere with the anti-cachectic actions of CCDTHT is suitable. The stock solution contains 1 to 5-mg CCDTHT per 1-ml. In some embodiments of the invention, 50-μl of a stock solution containing 2.25-mg CCDTHT per 1-ml is injected subcutaneously within 1-cm distance of the tumor. Considering that the weight of animals at the start of treatments were around 25 grams, this amount approximately corresponds to a dose of 4.5-mg per kg.

The treatments may start anytime between 7-20 days after tumor transplantation depending on the research goal. The treatments may be repeated daily or may be administered every second or third day. For maximum effectiveness, it is recommended that the treatments be performed at least every second day.

Tissue samples from the tumor, surrounding tissue or any other tissue may be taken for the analysis of biochemical and cellular changes at any time during the treatment and observation period. However, it is recommended that from one animal only one sample be taken.

This model provides an opportunity to compare biochemical and cellular changes in the tumor and other tissues in the untreated and CCDTHT-treated mice. Further, the set of changes induced by CCDTHT in this model can be compared to those obtained in the previous models using anti-cachectic proteins. Those changes that are commonly induced by the anti-cachectic proteins and CCDTHT are the most likely candidates to be involved in the development of cachexia.

Model 4. Treatment of Cisplatin-Treated MXT Mouse Mammary Tumors with Subcutaneously Injected Anti-Cachectic Proteins.

In this model, cisplatin-treated MXT mouse mammary tumors are treated with subcutaneously injected purified or commercial PALP or other protein components of the latter such as TF and AT alone or in combination with each other or purified PALP. In the MXT tumor model, cisplatin treatment causes significant reduction in body weight. Although the initiating mechanisms in case of tumor-induced and cisplatin-induced body weight loss probably differ, they likely to converge in a common mechanism which eventually executes cachexia. Comparison of biochemical and cellular changes in the tumor and other organs in cisplatin− and cisplatin+ protein-treated mice allows the investigator to draw conclusions about the mechanisms involved in cisplatin-induced cachexia. Comparison of data from this model with those derived from the previous models will allow to draw a conclusion whether chemotherapy- and tumor-induced cachexia involve similar, partially overlapping, or entirely different mechanisms.

In some embodiments, purified PALP is used. In other embodiments, commercial PALP as well as TF and AT are used. The proteins are suspended in a carrier and administered subcutaneously as described for Model 1.

Model 5. Treatment of Cisplatin-Treated MXT Mouse Mammary Tumors with Subcutaneously Injected CCDTHT.

In this model, cisplatin-treated MXT mouse mammary tumors are treated with subcutaneously injected CCDTHT compound that, similar to PALP, causes significant reversal of cisplatin-induced cachexia. This model will also provide a set of biochemical and cellular changes that are induced by cisplatin and prevented or reduced by CCDTHT in the tumor and healthy organs. Comparison of this set of changes with a similar set of changes obtained in Model 4 will provide essential help to sort out those common changes that are the most likely to directly relate to cisplatin-induced cachexia. Furthermore, comparison of data from this model with those obtained with all the other previous models will help to identify the mechanisms that are commonly involved in tumor- and chemotherapy-induced cachexia.

Preparation and administration of CCDTHT-containing solution in this model is performed as described for Model 3.

Model 6. Protein-Treated Mouse Skin Model to Study the Anti-Cachectic Signal(s) Generated by the Skin Tissue.

In this model, the future site of B16 melanoma tumor implantation in the mouse skin is pre-treated prior to the tumor implantation with commercial PALP-containing cream with no additional treatments performed after tumor implantation. As a result of such pre-treatments, tumor-bearing animals gained, instead of lost, body weight. This result indicates that commercial PALP can generate relatively long-lasting anti-cachectic signal(s) in the normal skin. Comparative analysis of untreated and protein-pretreated skin will facilitate identification of the anti-cachectic signal(s).

In Model 1, subcutaneously administered purified PALP and TF also significantly reversed tumor-induced body weight loss in the B16 melanoma model. Thus, it is reasonable to assume that these protein preparations also will exert anti-cachectic effects in this model. Accordingly, in this model, commercial PALP as well as purified PALP, TF and their combination may be used interchangeably to reverse tumor-induced cachexia.

Preparation and topical administration of protein-containing creams in this model is performed as described in Model 2. It is recommended that the site of future tumor implantation be treated at least twice, six and three days prior to implantation. More frequent and longer pre-treatments to further increase the anti-cactectic effects are within the scope of this model.

Factors

### EXAMPLES RELATING TO THE METHODS USED

### Example 1

### Purification and Spectrophotometric Assay of PALP

Human PALP (Type XXIV, 1020 units of total activity) in a partially purified form was obtained commercially from Sigma-Aldrich and was prepared by the method of Ghosh and Fishman [Ghosh, N. K. and Fishman, W. H. (1968), “Purification and properties of molecular-weight variants of human placental alkaline phosphatase,” Biochem. J., 108, 779-792]. Briefly, the purification steps described in that paper involve homogenization of human placenta in Tris, extraction with butanol, exposure to heat (55° C.), three successive precipitation of protein with ammonium sulfate followed by re-suspension, fractionation with ethanol twice, and Sephadex-G-200-gel filtration optionally followed by continuous curtain electrophoresis to further separate PALP variants.

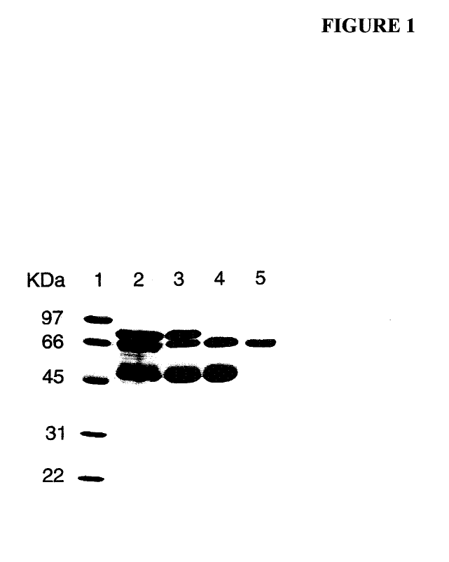

As determined by sodium dodecyl sulfate-polyacrylamide gel electrophoresis (SDS-PAGE) and peptide sequence analysis, the partially purified PALP obtained from Sigma-Aldrich (denoted “commercial PALP” herein) was not homogeneous and contained other proteins. FIG. 1 shows a picture of a gel separation of a preparation comprising commercial PALP without further purification, and other preparations of PALP of increasing purity. Separation of proteins was performed by conventional SDS-PAGE, and proteins were stained with coomassie blue stain. Lane 1 contains various molecular mass standards for comparison. Lane 2 represents a preparation containing commercial PALP with a strong 52 kDa band representing AT and another strong ˜66-68 kDa band representing a mixture of PALP and albumin. The upper, ˜80 kDa band is represented by TF. Lanes 3 and 4 represent preparations comprising commercial PALP material after further purification steps (described below), and lane 5 represents a preparation of homogeneous PALP obtained by the complete purification procedure described below.

A purification procedure was performed to further purify the commercially obtained PALP to homogeneity. A slightly modified procedure described earlier [She, Q.-B., Mukherjee, J. J., Huang, J.-S., Crilly, K. S. and Kiss, Z. (2000), “Growth factor-like effects of placental alkaline phosphatase in human and mouse embryo fibroblasts,” FEBS Lett., 469, 163-167] was used which is incorporated here by reference. The solution of commercial PALP was prepared by dissolving 350 mg of commercial PALP into 10 ml of buffer A (0.1 M sodium acetate, 0.5 M NaCl, 1 mM MgCl₂, 1 mM CaCl₂, adjusted to pH 6.5). This solution was then further purified by successive Concanavalin A-Sepharose and Q-Sepharose chromatography as indicated earlier by Chang et al. Chang, T.-C., Huang, S.-M., Huang, T.-M. and Chang, G.-G. (1992), “Human placenta alkaline phosphatase: An improved purification procedure and kinetic studies,” Eur. J. Biochem., 209, 241-247] followed by t-butyl hydrophobic interaction chromatography as described by She et al [She, Q.-B., Mukherjee, J. J., Huang, J.-S., Crilly, K. S. and Kiss, Z. (2000), “Growth factor-like effects of placental alkaline phosphatase in human and mouse embryo fibroblasts.” FEBS Lett., 469, 163-167] except that this last step was repeated in about 60% of cases (to eliminate traces of contaminant protein) in the invention.

First, the PALP solution was passed through a Concanavalin A-Sepharose column followed by an elution step using buffer A (50 mM α-methyl-D-mannopyranoside) as solvent. The active fractions collected from the effluent were pooled and dialyzed against buffer B (50 mM Tris-HCL at pH 7.7). SDS-PAGE separation of the collected and dialyzed fraction is shown in FIG. 1 in lane 3.

The collected and dialyzed fraction from the previous step was then passed through a Q-Sepharose column. The fraction of interest was eluted with buffer B using a linear gradient of 0-250 mM potassium phosphate at a pH of 7.5. The active fractions from the Q-Sepharose column were pooled and dialyzed against phosphate-buffered saline and concentrated by Amicon ultrafiltration. SDS-PAGE separation of the collected and dialyzed fraction is shown in FIG. 1 in lane 4, which demonstrates that at least two major proteins are still present in the fraction after dialysis.

Then, the collected and dialyzed fraction from the previous step was purified to homogeneity by t-butyl hydrophobic interaction chromatography (HIC). Prior to adding the fraction to the t-butyl HIC column, the fraction was made 2 M in ammonium sulfate, and the pH was adjusted to 6.8. The 5-ml bed volume t-butyl HIC cartridge (BIO-RAD, Hercules, Calif.) was connected to a fast performance liquid chromatography (FPLC) system from PHARMACIA (Peapack, N.J.). The fraction was introduced to the HIC column, and the column was eluted with buffer C (100 mM sodium phosphate buffer, 2 M ammonium sulfate at pH 6.8). The column was eluted with buffer C until a first protein-containing fraction completely eluted, and then a negative gradient of 2 M-to-0 M ammonium sulfate in 100 mM sodium phosphate at pH 6.8 was passed over the column. The negative linear gradient was used to elute a second protein-containing fraction, which contained the enzymatically active PALP protein.

The enzymatically active PALP fraction from the HIC separation was dialyzed against phosphate buffered saline and concentrated by Amicon ultrafiltration. The presence and purity of the PALP enzyme in the fraction was confirmed by SDS-PAGE. After electrophoretic separation, the gel was stained using coomassie blue or silver stain for visual observation of protein bands. When a single protein band with an approximate molecular weight of 66 kDa was not observed, the last chromatographic step was repeated. The pure PALP was further identified by sequence analysis performed by the Mayo Clinic Protein Core Facility (Rochester, Minn., US).

PALP enzyme activity was assayed using a spectroscopic method by monitoring the hydrolysis of 4-nitrophenylphosphate (as an increase in absorbance at 410 nm) at room temperature (22° C.) as described in Chang, G.-G., Shiao, M.-S., Lee, K.-R. and Wu, J.-J. (1990), “Modification of human placental alkaline phosphatase by periodate-oxidized 1,N⁶-ethenoadenosine monophosphate,” Biochem. J., 272, 683-690. Activity analysis of 5-10 μg purified enzyme was performed in 1 mL incubation volume containing 50 mM Na₂CO₃/NaHCO₃, 10 mM MgCl₂, 10 mM 4-nitrophenylphosphate at pH 9.8. The extinction coefficient of 4-nitrophenol was taken as 1.62×10⁴ M⁻¹cm⁻¹. An enzyme activity of 1 U (unit) is defined as 1 μmol substrate hydrolyzed/min at 22° C. at pH 9.8.

### Example 2

### Purification of AT

A partially purified human placental alkaline phosphatase preparation (PALP) was acquired from Sigma-Aldrich, Inc. AT is a major contaminant of the commercially obtained PALP. AT was first further purified by successive Concanavalin A-Sepharose and Q-Sepharose chromatography as described by Chang et al. for the isolation of PALP [Chang, T.-C., Huang, S.-M., Huang, T.-M. and Chang, G.-G. (1992), “Human placenta alkaline phosphatase: An improved purification procedure and kinetic studies,” Eur. J. Biochem., 209, 241-247]. The Q-Sepharose fraction, which still contained placental alkaline phosphatase in addition to AT, was further purified to homogeneity by t-butyl HIC chromatography [She, Q.-B., Mukherjee, J. J., Crilly, K. S. and Kiss, Z. (2000), “α₁-Antitrypsin can increase insulin-induced mitogenesis in various fibroblast and epithelial cell lines,” FEBS Lett., 473, 33-36]. The 5 ml bed volume t-butyl HIC cartridge was connected to a PHARMACIA FPLC system and the fractions containing AT were pooled. The purity was confirmed by SDS-PAGE (polyacrylamide gel electrophoresis) using coomassie blue stain. The purified protein was identified as AT by sequence analysis. The sequence analysis was performed by the Mayo Clinic Protein Core Facility (Rochester, Minn., USA). The protein concentration was determined by the Lowry assay, using bovine serum albumin as standard, with a protein assay kit from Sigma-Aldrich, Inc. according to the instructions. This purification procedure has been previously published [She, Q.-B., Mukherjee, J. J., Crilly, K. S. and Kiss, Z. (2000), “α₁-Antitrypsin can increase insulin-induced mitogenesis in various fibroblast and epithelial cell lines,” FEBS Lett., 473, 33-36].

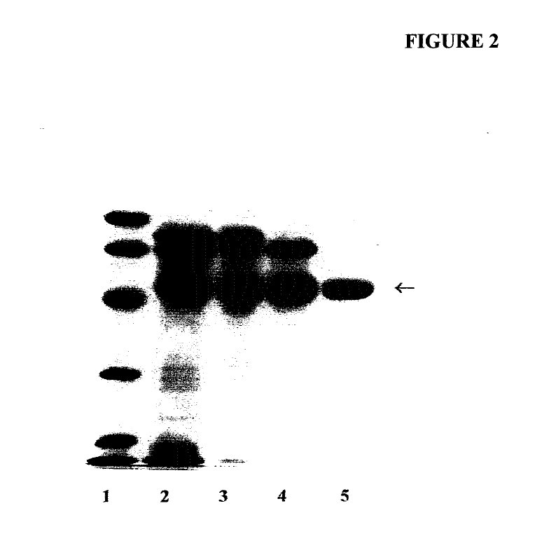

FIG. 2 is an image of a stained gel. The gel includes the commercially obtained partially purified placental alkaline phosphatase preparation (shown in lane 2) further purified by successive Concanavalin A-Sepharose (lane 3), Q-Sepharose (lane 4), and t-butyl HIC chromatography using 2 M-to-0 M ammonium sulfate gradient (lane 5). Lane 1 contains molecular mass standards of 97 kDa (top), 66 kDa, 45 kDa, 31 kDa, and 22 kDa (bottom) in that order. FIG. 2 demonstrates that while the commercially obtained preparation contains three major protein bands (one of them is represented by AT as indicated by the arrow, while a ˜66-68 kDa band represents placental alkaline phosphatase+albumin) and several minor proteins, the purified preparation contains only AT. The upper, about ˜80 kDa, band is represented by TF alone as determined by sequence analysis.

### Example 3

### Development and Treatment of Tumor Models

The B16 mouse melanoma and MXT mouse mammary tumors were developed in first generation hybrid BDF1 (C57 B1 female×DBA/2 male) adult female mice kept at specified pathogen free (SPF) hygienic level. These models were chosen for these experiments because these mice have the complete immune system, and both tumors grow very rapidly. To develop the tumors, B16 and MXT tumor tissue fragments of about 0.1-cm³ were surgically implanted subcutaneously into the intrascapular region to develop the tumors. Each tumor fragment contained about 1-1.5×10⁶ cells. The animals were kept in macrolon cages on ventilated rack at 22-24° C. (50-60% humidity) with lighting regimen of 12/12 h light/dark. The animals had free access to tap water and were fed with sterilized standard diet (Charles River VRF1, Germany) ad libitum. The animals were taken care of according to the “Guiding Principle for the care and use of Animals” based upon the Helsinki declaration and they were approved by the local ethical committee.

In case of both tumor models the tumor size was usually in the range of 0.2 to 0.5-g when treatments started 7-11 days after tumor implantation; the mice were selected so that in the same treatment group the difference in tumor size was no greater than 0.1 gram. In some experiments, the test proteins were suspended in physiological saline and injected in 50-μl volume subcutaneously in the vicinity of tumor (within 1-cm of tumor margin). In other experiments, the test proteins were dispersed in Vaselinum cholesteratum and used topically; 150-mg cream was applied on the tumor and the vicinity of tumor area (with about 0.5-cm of the tumor margin). The tumor volume was determined by using calipers; this technique is well known to one having ordinary skill in the art. Tumor volume was calculated according to the generally accepted formula: V=a²×b×7r/6, where “a” and “b” mean the shortest and longest diameter, respectively, of the measured tumor.

### Example 4

### Effects of Subcutaneously Injected Highly Purified or Commercial PALP on the Body Weights of B16 Melanoma Bearing Mice. Model 1

In this model, either highly purified PALP or commercial PALP was subcutaneously injected to reverse body weight loss in the rapidly growing B16 melanoma model. Two separate experiments were performed to validate the effects of highly purified PALP (Experiment 1) and commercial PALP (Experiment 2). For both experiments, the B16 mouse melanoma model was developed as described in Example 3. The data presented in TABLE 1 (for Experiment 1) and TABLE 2 (for Experiment 2) show the changes in total weight, tumor weight, and body weight (total weight minus tumor weight). In each experimental group all mice survived until the conclusion of the experiment.

Experiment 1. In the first experimental group, from day 11 (11 days after tumor implantation) until the end of experiment (day 21) the animals remained untreated. In the second experimental group, the animals received 14-mg/kg of highly purified PALP on days 11, 13, 15 and 17 by subcutaneous (s.c.) injection. In both groups five mice were included.

As shown in TABLE 1, in this experimental model administration of purified PALP by injection reversed loss of body weight even though at this concentration it had no major effects on the tumor size. The modest gain in body weight in the treated animals is an indication that injection of highly purified PALP can effectively neutralize the cachectic effect of B16 melanoma tumor. This means that the tumor-generated cachexia-inducing biochemical and cellular changes are reduced or suppressed by injected purified PALP. Thus, the purified PALP-treated B16 mouse melanoma model is suitable to study the tumor-generated biochemical and cellular mechanisms involved in tumor-induced cachexia.

TABLE 1
Subcutaneously injected highly purified PALP prevents
body weight loss in the B16 melanoma model.

| | | Days after tumor transplantation | | | | |
|---|---|---|---|---|---|---|
| Compounds | Weight (g) | Day 11 | Day 14 | Day 16 | Day 18 | Day 21 |
| Untreated | Total | 22.60 | 22.80 | 22.90 | 23.30 | 23.10 |
| | weight | | | | | |
| | Tumor | 0.60 | 0.90 | 1.35 | 2.00 | 2.90 |
| | weight | | | | | |
| | Body | 22.00 | 21.90 | 21.55 | 21.30 | 20.20 |
| | weight | | | | | |
| PALP | Total | 22.00 | 22.80 | 23.80 | 25.70 | 26.90 |
| | weight | | | | | |
| | Tumor | 0.55 | 0.60 | 1.10 | 1.90 | 2.40 |
| | weight | | | | | |
| | Body | 21.45 | 22.20 | 22.70 | 23.80 | 24.50 |
| | weight | | | | | |

Experiment 2. In this experiment, the first group of animals remained untreated for the entire observation period. In the second group, from day 11 through day 18 after implantation of tumor pieces, the animals were treated daily with 60-mg/kg of commercial PALP via s.c. administration (seven treatments). In the third group, from day 11 through day 18 after implantation of tumor pieces, the animals were simultaneously administered 60-mg/kg of commercial PALP via s.c injection and treated topically with 150-mg of cream containing 4-mg commercial PALP in 1-g of Vaselinum cholesteratum (seven treatments). Topical treatment involved spreading the cream on both the tumor and surrounding skin area. In the fourth group, from day 11 through day 18 after implantation of tumor pieces, the animals were treated with 16-mg/kg of commercial partially iron-saturated TF via subcutaneous administration (seven treatments). Each group included seven mice.

The results in TABLE 2 show that s.c. injected commercial PALP also reverses body weight loss. Local administration of commercial PALP only slightly improved the effect of injected commercial PALP. Subcutaneous administration of TF, which constitutes about 12% of commercial PALP, stabilized body weights but did not promote weight gain. Overall, the experiments presented in TABLE 1 and TABLE 2 indicate that in the B16 tumor model s.c. injected highly purified and commercial PALP are similarly suitable to reverse cachexia and thereby allow the study of tumor-generated cachexia-inducing biochemical and cellular mechanisms. Any of the biochemical and cellular mechanisms occurring in the tumor or any of the other tissues may be studied. By example only, the studied mechanisms may involve alterations in oncogene and cytokine expression in the tumor, protein metabolism in the muscle, and lipid metabolism in the adipose tissue. The potential number of biochemical and cellular studies is similarly unlimited in case of all the other presented models; to simplify the text, this fact will not be repeated at the description of the other models.

TABLE 2
Subcutaneously administered commercial PALP also
reverses body weight loss in B16 melanoma bearing mice.

| | | Days after tumor transplantation | | | | | |
|---|---|---|---|---|---|---|---|
| Treatment | Data in gram | 11 | 14 | 16 | 18 | 21 | 23 |
| None | Total weight | 23.9 | 24.3 | 24.6 | 24.9 | 25.3 | 25.7 |
| | Tumor weight | 0.6 | 1.0 | 1.5 | 2.1 | 3.2 | 4.1 |
| | Body weight | 23.3 | 23.3 | 23.1 | 22.8 | 22.1 | 21.6 |
| PALP s.c. | Total weight | 23.0 | 24.4 | 25.9 | 26.5 | 27.7 | 28.0 |
| | Tumor weight | 0.5 | 0.8 | 1.1 | 1.5 | 2.4 | 2.5 |
| | Body weight | 22.5 | 23.6 | 24.8 | 25.0 | 25.3 | 25.5 |
| PALP s.c. + | Total weight | 23.1 | 24.6 | 25.3 | 26.0 | 27.9 | 28.3 |
| PALP | Tumor weight | 0.6 | 0.6 | 0.7 | 0.9 | 1.5 | 2.0 |
| topical | Body weight | 22.5 | 24.0 | 24.6 | 25.1 | 26.4 | 26.3 |
| TF s.c. | Total weight | 22.9 | 24.2 | 24.3 | 25.0 | 26.1 | 26.6 |
| | Tumor weight | 0.5 | 0.7 | 0.8 | 1.4 | 2.2 | 2.7 |
| | Body weight | 22.4 | 23.5 | 23.5 | 23.6 | 23.9 | 23.9 |

### Example 5

### Topical Treatment with Commercial PALP Reverses Loss of Body Weight in the Mouse B16 Melanoma Tumor Model. Model 2

The B16 mouse melanoma model was developed as described under Example 3. In the first group, mice remained untreated during the whole observation period. In the second group, after 8 days of tumor implantation the tumor along with the surrounding skin was treated with Vaselinum cholesteratum alone (150-mg per application). In the third group, after 8 days of tumor implantation the tumor along with the surrounding skin was treated with 150-mg of cream containing 4-mg of commercial PALP in 1-g of Vaselinum cholesteratum. Animals in the second and third groups were also treated on days 11, 13, 15, 18 and 20 after tumor implantation. Each group included 5 animals. The mean values for total weight, tumor weight and body weight (the difference between total and tumor weight) are shown in TABLE 3.

The results show that untreated animals or animals treated only with Vaselinum cholesteratum did not gain, in fact lost, body weight during the 12-day observational period. In contrast, treatment of animals with commercial PALP 6-times increased their body weight from 22.5-g on day 8 to 28.1-g on day 20. This rate approximately corresponds to normal body weight growth experienced during the same period by tumor-free mice of similar age (not shown). Thus, in this experimental tumor model frequent topical administration of commercial PALP on the tumor and surrounding skin is suitable to maintain body weight growth. Based on the previous findings that injected purified PALP and injected commercial PALP had comparable effects on the body weight in this model, it is reasonable to expect that topical application of purified PALP will also result in significant reversal of body weight loss. Since commercial PALP also contains TF, AT and albumin, preparations that contain any of these or all of these proteins in addition to PALP should be suitable to decrease tumor-induced body weight loss in this B16 mouse melanoma model.

TABLE 3
Topically administered commercial PALP increases
body weight in B16 melanoma bearing mice.

| | | Days after tumor transplantation | | | | | |
|---|---|---|---|---|---|---|---|
| Treatment | Data in gram | 8 | 11 | 13 | 15 | 18 | 20 |
| None | Total weight | 22.7 | 23.7 | 24.2 | 24.6 | 25.0 | 25.2 |
| | Tumor weight | 0.3 | 0.6 | 1.2 | 1.9 | 2.6 | 3.5 |
| | Body weight | 22.4 | 23.1 | 23.0 | 22.7 | 22.4 | 21.7 |
| Vaseline | Total weight | 23.0 | 24.1 | 24.8 | 25.0 | 25.8 | 26.0 |
| alone | Tumor weight | 0.3 | 0.7 | 1.1 | 1.7 | 2.7 | 3.5 |
| | Body weight | 22.7 | 23.4 | 23.7 | 23.3 | 23.1 | 22.5 |
| PALP | Total weight | 22.8 | 24.2 | 25.0 | 25.6 | 28.0 | 30.1 |
| | Tumor weight | 0.3 | 0.5 | 0.6 | 1.0 | 1.6 | 2.0 |
| | Body weight | 22.5 | 23.7 | 24.4 | 24.8 | 26.4 | 28.1 |

### Example 6

### Determination of Cytokine mRNA Expression in B16 Tumors that were Either Untreated or Topically Treated with PALP

The goal in this experiment was demonstrate that topically applied PALP cream is capable of inducing significant biochemical events in the tumor. Cytokine mRNA expression was chosen because certain cytokines may be involved both in the induction and repression of cachexia. In the experiment reported in TABLE 4, mRNA expression was determined for the following cytokines: interleukin-1α (IL-1α), interleukin-1β (IL-1β), interleukin-6 (IL-6) and tumor necrosis factor-α (TNFα).

The B16 mouse melanoma model was developed as described in Example 3. In the first group, mice remained untreated during the whole observation period. In the second group, after 8 days of tumor implantation the tumor along with the surrounding skin was treated with 150-mg of cream containing 4-mg of commercial PALP in 1-g of Vaselinum cholesteratum. Then the treatments were repeated 11, 13, 15, 18 and 20 after tumor implantation. One day after the second, fourth and sixth applications tumor tissue was collected for analysis. Each group initially included 9 animals. All data presented in TABLE 4 are derived from 3 separate determinations with each sample being taken from separate tumors. Each mouse used only once for sample collection.

For the determination of mRNA expression, the samples were homogenized and total RNA was prepared using Magna Pure LC (Roche). RNA (250-ng) was reverse-transcribed (Sigma, Enhanced Avian Reverse Transcriptase-PCR kit) according to the manufacturer's instructions. The reverse transcription-polymerase chain reaction (RT-PCR) was performed by the Light Cycler 2.0 instrument using the following conditions:

IL-1α. Denaturation: 95° C., 5 sec; annealing: 63° C., 3 sec; elongation: 72° C., 6 sec.

IL-1β. Denaturation: 95° C., 5 sec; annealing: 63° C., 3 sec; elongation: 72° C., 6 sec.

IL-6. Denaturation: 95° C., 5 sec; annealing: 60° C., 3 sec; elongation: 72° C., 5 sec.

TNFα. Denaturation: 95° C., 5 sec; annealing: 62° C., 5 sec; elongation: 72° C., 7 sec.

The results, shown in TABLE 4, are expressed as percentage of glyceraldehyde phosphate dehydrogenase (GAPDH) expression which is taken as 100%. Expression of GAPDH, called a housekeeping gene, is taken as reference (a widely accepted method) because it is remarkably constant under various physiological conditions. The results indicate that topically applied commercial PALP preparation significantly increases the expression of mRNA for tumor TNFα, IL-1β and IL-6 and to a smaller extent that of IL-1α. Most changes were evident after the second application with only relatively small additional changes occurring between the second and 6th applications. Overall, the experiment proves that topically applied commercial PALP significantly affects the biochemical and cellular events in the tumor. It is reasonable to assume that topically applied purified PALP and TF would similarly affect tumor biology.

TABLE 4
Stimulatory effect of commercial PALP on the
expression of cytokines in B16 melanoma after 2, 4 and 6 treatments.

| | Cytokine mRNA expression relative to GAPDH expression (100%) | | | | | | | | | | | |
|---|---|---|---|---|---|---|---|---|---|---|---|---|
| | IL-1α | | | IL-1β | | | IL-6 | | | TNF-α | | |
| Treatment | 2^{nd} | 4^{th} | 6^{th} | 2^{nd} | 4^{th} | 6th | 2^{nd} | 4^{th} | 6th | 2^{nd} | 4^{th} | 6th |
| Vehicle | — | — | 82 | — | — | 81 | | | 95 | — | — | 82 |
| PALP | 109 | 124 | 122 | 165 | 187 | 210 | 169 | 175 | 180 | 178 | 195 | 208 |

### Example 7

### Synthesis of N,N-diethyl-N-methyl-2-[(9-oxo-9H-thioxanthen-2-yl)methoxy]-ethanaminium iodide (CCDTHT)

This has been performed by using the procedure described in a filed U.S. patent application [U.S. patent application Ser. No. 11/458,502, filed on Jul. 19, 2006 and entitled “Compounds and compositions to control abnormal cell growth”].

### Example 8

### CCDTHT Reverses Tumor-Induced Body Weight Loss in B16 Melanoma-Bearing Mice. Model 3

In this model, CCDTHT, a chemically synthesized compound is used to prevent or reverse tumor-induced body weight loss. The B16 mouse tumors were developed as described in Example 3. Two experimental groups each involving five mice were used. In the first group, mice remained untreated during the entire observation period. In the second group, starting on the 11th day after tumor implantation, mice were s.c. administered 4.5-mg/kg CCDTHT; then the treatments were repeated daily until day 22. The mean values for total weight, tumor weight and body weight (the difference between total and tumor weight) are shown in TABLE 5.

The results show that while control (untreated) animals gradually lost weight from day 14, CCDTHT-treated animals experienced modest weight gain. For example, on day 23 CCDTHT-treated mice were 2.6 grams heavier than control mice (about 10% difference).

The significance of this model is that it allows assessment of the role of various tumor-induced biochemical and cellular changes in the development of cachexia independent of PALP and PALP-containing preparations. Tumor-induced changes that are prevented or reduced by CCDTHT are likely to be involved in cachexia. Moreover, changes seen with both PALP- and CCDTHT-treated B16 melanoma-bearing mice are the most likely to relate to tumor-induced cachexia.

TABLE 5
CCDTHT reverses tumor-induced body weight loss in
the B16 melanoma model.

| | | Days after tumor transplantation | | | | | |
|---|---|---|---|---|---|---|---|
| Treatments | Weight (g) | Day 11 | Day 14 | Day 16 | Day 18 | Day 21 | Day 23 |
| Untreated | Total weight | 22.9 | 23.3 | 23.3 | 23.8 | 24.5 | 25.1 |
| | Tumor weight | 0.5 | 0.9 | 1.4 | 2.0 | 2.9 | 3.9 |
| | Body weight | 22.4 | 22.4 | 21.9 | 21.8 | 21.6 | 21.2 |
| CCDTHT | Total weight | 23.0 | 23.7 | 24.2 | 24.7 | 25.4 | 25.9 |
| | Tumor weight | 0.6 | 0.8 | 1.0 | 1.3 | 1.8 | 2.1 |
| | Body weight | 22.4 | 22.9 | 23.2 | 23.4 | 23.6 | 23.8 |

### Example 9

### PALP and TF Prevents Cisplatin-Induced Body Weight Loss in the MXT Tumor Model. Model 4

Experiment 1. The mouse MXT mammary tumor was developed as described in Example 3. In the first group, animals remained untreated during the whole observation period. In the second group, starting on the 7th day after tumor implantation, mice were intraperitoneally (i.p.) administered 3-mg per 1-kg total weight of cisplatin 7-times every second day. In the third group, starting on the 7th day after tumor implantation, mice were simultaneously administered 3-mg per 1-kg of cisplatin i.p. and 16-mg per 1-kg of purified PALP s.c. In the fourth group, starting on the 7th day after tumor implantation, mice were simultaneously administered 3-mg per 1-kg of cisplatin i.p. and 60-mg per 1-kg of commercial PALP s.c. In the fifth group, starting on the 7th day after tumor implantation, mice were simultaneously administered 3-mg per 1-kg of cisplatin i.p. and 14-mg per 1-kg of commercial TF (T 3309 in the Sigma catalog) s.c. Each treatment was performed 7-times on every second day. Each group included 7 animals. The mean values for total weight, tumor weight and body weight (the difference between total and tumor weight) are shown in TABLE 6.

The results indicate that in this tumor model, cisplatin induced dramatic (˜33%) decrease in body weight from day 11 to day 23. Purified PALP not only prevented the loss but significantly enhanced body weight. On day 21 after tumor transplantation, the body weight of mice co-treated with cisplatin and purified PALP was 9.6 grams more than that of mice treated with cisplatin alone and 4.8 grams more than that of untreated mice. Commercial PALP also reversed, although somewhat less effectively than purified PALP, body weight loss induced by the tumor and cisplatin. Finally, partially iron-saturated commercial TF also partially reversed body weight loss induced by the tumor and cisplatin.

It is reasonable to predict that in the MXT tumor model, tumor- or chemotherapy-induced biochemical and cellular changes (up or down) in the tumor and other tissues that are altered to the opposite by injected purified or commercial PALP or TF are likely to contribute to the development of cachexia. Thus, the data presented in TABLE 6 provide a reasonably solid basis to use the mammary MXT tumor model, treated with TF, commercial PALP or highly purified PALP, to study the biochemical and cellular events associated with tumor- and/or chemotherapy-induced body weight loss. In view of the larger magnitude of body weight loss upon chemotherapy, this model is particularly useful to study the mechanisms by which chemotherapy induces cachexia.

TABLE 6
Comparison of reversal effects PALP, TF and AT on
cisplatin-induced loss of body weight in the mouse MXT mammary tumor model.

| | | Days after tumor transplantation | | | | | |
|---|---|---|---|---|---|---|---|
| | | | | Day | Day | Day | Day |
| Treatments | Weight (g) | Day 7 | Day 11 | 14 | 16 | 18 | 21 |
| Untreated (None) | Total weight | 22.30 | 24.50 | 25.40 | 26.70 | 26.70 | 27.70 |
| | Tumor weight | 0.25 | 2.05 | 4.10 | 5.60 | 6.40 | 7.80 |
| | Body weight | 22.05 | 22.45 | 21.30 | 21.10 | 20.30 | 19.90 |
| Cisplatin | Total weight | 22.70 | 23.10 | 21.70 | 20.10 | 20.10 | 19.20 |
| | Tumor weight | 0.25 | 1.00 | 2.20 | 3.20 | 3.80 | 4.10 |
| | Body weight | 22.45 | 22.10 | 19.50 | 16.90 | 16.30 | 15.10 |
| Cisplatin + purified | Total weight | 22.10 | 24.20 | 25.40 | 26.00 | 26.00 | 22.70 |
| PALP | Tumor weight | 0.20 | 0.80 | 1.80 | 2.30 | 2.90 | 3.00 |
| | Body weight | 21.90 | 23.40 | 23.60 | 23.70 | 23.00 | 24.70 |
| Cisplatin + commerc. | Total weight | 22.20 | 25.10 | 26.40 | 25.60 | 25.60 | 26.60 |
| PALP | Tumor weight | 0.25 | 1.25 | 2.70 | 3.60 | 3.75 | 3.80 |
| | Body weight | 21.95 | 23.85 | 23.70 | 22.00 | 21.85 | 22.80 |
| Cisplatin + TF | Total weight | 21.80 | 24.10 | 24.60 | 24.90 | 24.90 | 25.50 |
| | Tumor weight | 0.25 | 0.75 | 1.50 | 2.15 | 2.90 | 3.65 |
| | Body weight | 21.55 | 23.35 | 23.10 | 22.75 | 22.00 | 21.85 |

Experiment 2. The commercial PALP used in this model for Exp. 1 was composed of PALP (˜10%), TF (˜12%), AT (˜30%), albumin (˜35%) and the degradation products of TF (˜13%). In separate experiments human albumin, injected s.c. in the amount 16-mg per 1-kg did not alter body weight in cisplatin-treated MXT tumor-bearing animals (data not shown). In this experiment, it was determined whether the other two major proteins, i.e. TF and AT, present in the commercial PALP preparation in addition to PALP, also influence cisplatin-induced loss of body weight or not. The mouse MXT mammary tumor was developed as described in Example 3. In the first group, animals remained untreated during the whole observation period. In the second group, starting on the 7th day after tumor implantation, mice were administered 3-mg per 1-kg of cisplatin i.p. 6-times every second day. In the third group, starting on the 7th day after tumor implantation, mice were simultaneously administered 3-mg per 1-kg of cisplatin i.p. and 12-mg per 1-kg of purified PALP s.c. In the fourth group, starting on the 7th day after tumor implantation, mice were simultaneously administered 3-mg per 1-kg of cisplatin i.p. and 12-mg per 1-kg of highly purified AT s.c. In the fifth group, starting on the 7th day after tumor implantation, mice were simultaneously administered 3-mg per 1-kg of cisplatin i.p. and 12-mg per 1-kg of commercial holo-TF (h-TF, T 0665 in the Sigma catalog) s.c. In the sixth group, starting on the 7th day after tumor implantation, mice were simultaneously administered 3-mg per 1-kg of cisplatin i.p. and 12-mg per 1-kg of commercial apo-TF (a-TF, T 2036 in the Sigma catalog) s.c. Holo-TF contained 1,100-1,600 μg iron per gram protein while apo-TF was essentially iron-free. Each treatment with the proteins was also performed 6-times on every second day simultaneously with the cisplatin treatment. Each group included 7 animals. The mean values for total weight, tumor weight and body weight (the difference between total and tumor weight) are shown in gram in TABLE 7.

The results indicate that AT, holo-TF and apo-TF each partially reversed cisplatin-induced body weight loss, however, these proteins are less effective than purified PALP. Overall, the data indicate that both TF and AT may contribute to the anti-cachectic effects of commercial PALP, although PALP is clearly the most effective component. Based on the data presented in TABLE 7, this model allows the use of TF (both iron-free and iron-containing) and AT in combination with each other and/or purified PALP to reverse or reduce chemotherapy-induced body weight loss.

TABLE 7
Comparison of effects of purified PALP, TF and AT on
the body weight in the Cispl.-treated MXT tumor model.

| | | Days after transplantation | | | | |
|---|---|---|---|---|---|---|
| | | Day | Day | Day | | |
| Treatment | Weight (g) | 7 | 11 | 13 | Day 15 | Day 18 |
| Untreated | Total weight | 22.6 | 24.4 | 25.0 | 26.3 | 27.5 |
| | Tumor weight | 0.4 | 2.6 | 4.1 | 5.45 | 7.6 |
| | Body weight | 22.2 | 21.8 | 20.9 | 20.85 | 19.9 |
| Cispl | Total weight | 22.6 | 22.7 | 21.9 | 20.8 | 20.3 |
| | Tumor weight | 0.35 | 1.45 | 1.85 | 2.7 | 3.5 |
| | Body weight | 22.25 | 21.25 | 20.05 | 18.1 | 16.8 |
| Cispl + PALP | Total weight | 22.3 | 22.7 | 23.0 | 23.9 | 24.4 |
| | Tumor weight | 0.4 | 1.3 | 1.7 | 2.55 | 2.95 |
| | Body weight | 21.9 | 21.4 | 21.3 | 21.35 | 21.45 |
| Cispl + AT | Total weight | 22.1 | 23.6 | 23.8 | 23.5 | 23.6 |
| | Tumor weight | 0.35 | 1.65 | 2.85 | 3.5 | 4.35 |
| | Body weight | 21.75 | 21.95 | 20.95 | 20.0 | 19.25 |
| Cispl + h-TF | Total weight | 22.8 | 22.8 | 22.7 | 22.8 | 23.5 |
| | Tumor weight | 0.45 | 1.45 | 2.5 | 2.9 | 4.1 |
| | Body weight | 22.35 | 21.35 | 20.2 | 19.9 | 19.4 |
| Cispl + a-TF | Total weight | 22.9 | 23.7 | 23.0 | 23.0 | 23.8 |
| | Tumor weight | 0.4 | 1.6 | 2.3 | 3.1 | 3.9 |
| | Body weight | 22.5 | 22.1 | 20.7 | 19.9 | 19.9 |

### Example 10

### CCDTHT Prevents Cisplatin-Induced Body Weight Loss in the MXT Tumor Model. Model 5

The mouse MXT mammary tumor was developed as described in Example 3. In the first group, animals remained untreated during the whole observation period. In the second group, starting on the 7th day after tumor implantation, mice were administered 3-mg per 1-kg of cisplatin i.p. 9-times every second day. In the third group, starting on the 7th day after tumor implantation, mice were simultaneously administered 3-mg per 1-kg of cisplatin i.p. and 4.6-mg per 1-kg of CCDTHT s.c. In the fourth group, starting on the 7th day after tumor implantation, mice were simultaneously administered 3-mg per 1-kg of cisplatin i.p. as well as 4.6-mg per 1-kg of CCDTHT s.c. and 14-mg per 1-kg of highly purified PALP s.c. Each treatment was performed 9-times on every second day. Each group included 7 animals. The mean values for total weight, tumor weight and body weight (the difference between total and tumor weight) are shown in TABLE 8.

The results indicate that CCDTHT effectively prevents cisplatin-induced body weight loss in the MXT tumor model. In fact, in the co-presence of CCDTHT and cisplatin, animals did not lose any weight; by the 21st day, these mice weighed 4.55 grams more than untreated mice and 7.15 grams more than mice treated with cisplatin alone. Co-treatment with CCDTHT and PALP was even more effective than CCDTHT alone to reverse body weight loss. Thus, treatment of cisplatin-treated MXT tumor with CCDTHT is suitable to the mechanisms involved in chemotherapy-induced cachexia. To achieve an even better effect, CCDTHT may be combined with PALP in this model.

TABLE 8
CCDTHT prevents body weight loss induced by MXT
tumor and cisplatin.

| | | Days after transplantation | | | | | |
|---|---|---|---|---|---|---|---|
| | | | | Day | Day | Day | Day |
| Treatment | Weight (g) | Day 7 | Day 9 | 11 | 15 | 17 | 21 |
| Untreated | Total weight | 22.40 | 23.60 | 24.05 | 25.60 | 26.70 | 27.40 |
| | Tumor weight | 0.25 | 0.70 | 1.55 | 4.20 | 5.40 | 8.85 |
| | Body weight | 22.15 | 22.90 | 22.50 | 21.40 | 21.30 | 18.55 |
| Cispl | Total weight | 22.20 | 23.00 | 23.10 | 22.90 | 21.60 | 20.60 |
| | Tumor weight | 0.25 | 0.55 | 1.35 | 2.65 | 3.75 | 4.65 |
| | Body weight | 21.95 | 22.45 | 21.75 | 20.25 | 17.85 | 15.95 |
| Cispl + CCDTHT | Total weight | 22.50 | 23.10 | 23.80 | 24.10 | 24.90 | 26.70 |
| | Tumor weight | 0.30 | 0.55 | 1.15 | 2.20 | 3.30 | 3.60 |
| | Body weight | 22.20 | 22.55 | 22.65 | 21.90 | 21.60 | 23.10 |
| Cispl + PALP + CCDTHT | Total weight | 22.00 | 23.20 | 23.60 | 25.40 | 25.40 | 27.90 |
| | Tumor weight | 0.30 | 0.50 | 0.70 | 1.70 | 2.25 | 2.60 |
| | Body weight | 21.70 | 22.70 | 22.90 | 23.70 | 23.15 | 25.30 |

### Example 11

### Pre-Treatment of Mouse Skin with PALP to Reverse Loss of Body Weight in the Mouse B16 Melanoma Tumor Model. Model 6

The experimental work for this Example and Example 2 was performed at the same time. However, the results that provide the basis for these separate models are presented separately for clarity. In the first group, mice remained untreated during the whole observation period; the data for this group are identical with data presented for the untreated group in TABLE 3. In the second group, the skin representing the future site of tumor implantation was treated with 150-mg of cream containing 4-mg of commercial PALP in 1-g of Vaselinum cholesteratum 6 and 3 days prior to the implantation. Both groups included 5 animals. The mean values for total weight, tumor weight and body weight (the difference between total and tumor weight) are shown in TABLE 9.

The results clearly indicate that while control animals progressively lost body weight starting on the 13th day after tumor transplantation, PALP-pretreated animals actually gained body weight over the entire observation period. For example, after 20 days of tumor implantation, the difference in body weight between the control and PALP-treated group was 4.3 grams in favor of the latter group. Again, based on the previous findings that injected purified PALP and injected commercial PALP had comparable effects on the body weight in the B16 model, it is reasonable to expect that pretreatment of skin with purified PALP would also lead to a significant reversal of body weight loss. Since commercial PALP also contains TF, AT and albumin, pretreatment of skin with preparations that contain any of these proteins or all of these proteins in addition to PALP should be suitable to decrease tumor-induced body weight loss in the B16 mouse melanoma model.

TABLE 9
Pretreatment of skin with commercial PALP reverses tumor-induced
decrease in body weight in B16 melanoma bearing mice.

| | | Days after tumor transplantation | | | | | |
|---|---|---|---|---|---|---|---|
| Treatment | Data in gram | 8 | 11 | 13 | 15 | 18 | 20 |
| None | Total weight | 22.7 | 23.7 | 24.2 | 24.6 | 25.0 | 25.2 |
| | Tumor weight | 0.3 | 0.6 | 1.2 | 1.9 | 2.6 | 3.5 |
| | Body weight | 22.4 | 23.1 | 23.0 | 22.7 | 22.4 | 21.7 |
| PALP | Total weight | 23.2 | 24.5 | 25.5 | 26.1 | 28.3 | 29.0 |
| | Tumor weight | 0.2 | 0.5 | 0.9 | 1.5 | 2.2 | 3.0 |
| | Body weight | 23.0 | 24.0 | 24.6 | 24.6 | 26.1 | 26.0 |

1. A mouse model to identify biochemical and cellular mechanisms causally relating to unintentional weight loss or cachexia comprising:
providing a first mouse and a second mouse having an implanted cachexia-inducing tumor;
treating the first and the second mouse with established weight loss-inducing cancer therapies
treating the second mouse with one or more established anti-cachectic or weight loss reducing agents selected from the group consisting of placental alkaline phosphatase, transferrin, α1-antitrypsin, N,N-diethyl-N-methyl-2[(9-oxo-9H-thioxanthen-2-yl)methoxy] ethanaminium iodide and combinations thereof to substantially reduce the cachexia symptoms,
selecting suitable biochemical and cellular mechanism for analysis; and
comparing biochemical or cellular changes between the first and second mouse.

2. (canceled)

3. The mouse model of claim 2 wherein the weight loss-inducing cancer therapy is chemotherapy.

4. The mouse model of claim 3 wherein the chemotherapy is provided by cisplatin.

5. The mouse model of claim 1 wherein the weight loss-inducing cancer therapy is radiation therapy or immunotherapy.

6. The mouse model of claim 1 wherein the tumor is a B16 mouse melanoma or MXT mouse mammary tumor.

7-8. (canceled)

9. A method for identifying the biochemical and cellular mechanisms causally relating to unintentional weight loss or cachexia comprising:
implanting a B16 mouse melanoma or a MXT mouse mammary tumor into a first and second mouse;
treating first and second mouse with established weight loss-inducing therapies:
administering an established anti-cachectic or weight loss reducing agent selected from the group consisting of placental alkaline phosphatase, transferrin, α1-antitrypsin, N,N-diethyl-N-methyl-2[(9-oxo-9H-thioxanthen-2-yl)methoxy] ethanaminium iodide and combinations thereof to the second mouse;
selecting suitable biochemical and cellular mechanism for analysis;
identifying biochemical or cellular changes induced by the tumor in the first mouse;
identifying the biochemical or cellular changes induced by the tumor and modified by the anti-cachectic agent in the second mouse; and
comparing the biochemical or cellular changes between the first and second mouse.

10. (canceled)

11. The method of claim 9 wherein the weight loss-inducing cancer therapy is chemotherapy.

12. The method of claim 11 wherein the chemotherapy is provided by cisplatin.

13. The method of claim 9 wherein the weight loss-inducing cancer therapy is radiation therapy or immunotherapy.

14. The method of claim 9 further comprising comparing the biochemical or cellular changes between the first and second mouse implanted with the B16 mouse melanoma tumor and the first and second mouse implanted with the MXT mouse mammary tumors.

15-16. (canceled)

17. The method of claim 9 wherein the administering is by injection, topical application or by a combination of injection and topical application.

18. The method of claim 9 wherein identifying biochemical or cellular changes includes changes in one of the following classes of endogenous factors comprising cytokines, regulators of food cycle, growth factors, steroids, lipases, mitochondria uncoupling proteins, proteases, regulators of metabolism such as proteolysis and lipid degradation, regulators of cell cycle, regulators of cell survival and differentiation, regulators of gene expression, transcription factors, regulators of protein, lipid and carbohydrate synthesis, glycocorticoids, metabolites and enzymes.

19. The method of claim 18 wherein the cytokines comprise tumor necrosis factor-α, interleukin-6, interleukin-1β, interleukin-1α, interleukin-12, other interleukins, or interferons.

20. The method of claim 18 wherein the regulators of food cycles comprise neuropeptide Y, leptin or gherlin.

21. The method of claim 18 wherein the growth factors comprise angiotensin II or insulin-like growth factor 1.

22. The method of claim 18 wherein the steroids comprise testosterone.

23. The method of claim 18 wherein the lipases comprise lipoprotein lipase.

24. The method of claim 18 wherein the mitochondria uncoupling proteins comprise uncoupling proteins 1, 2 or 3.

25. The method of claim 18 wherein the transcription factors comprise MyoD or nuclear factor kappa B.

26-27. (canceled)

28. The method of claim 18 wherein the metabolites comprise triacylglycerol and free fatty acids.

29. The method of claim 18 wherein the protease is an ATP-dependent ubiquitin-proteasome in the proteolytic pathway.

30. The method of claim 18 wherein proteolysis and lipid degradation regulators are proteolysis-inducing factor (PIF) and lipid-mobilizing factor zinc α2-glycoprotein.
